(19) United States
(12) Patent Application Publication
(10) Pub. No.: US 2004/0235850 A1
(43) Pub. Date: Nov. 25, 2004

(54) Pharmaceutical compositions of varenicline

(75) Inventor: Kenneth C. Waterman, East Lyme, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/848,464

(22) Filed: May 18, 2004

Related U.S. Application Data
(60) Provisional application No. 60/472,098, filed on May 20, 2003.

(51) Int. Cl. A61K 31/498

(52) U.S. Cl. 514/249

(57) ABSTRACT

The invention relates to novel pharmaceutical dosage forms of varenicline, which are useful for aiding smoking cessation and which have good storage stability. In particular, the present invention relates to formulations of varenicline wherein the dosage forms that are produced therefrom generate under specified storage conditions less than about 4% on a weight basis of the N-formyl and N-methyl degradation products.

### BACKGROUND OF THE INVENTION

The present invention is directed to novel pharmaceutical dosage forms of varenicline, a drug which binds to neuronal nicotinic acetylcholine specific receptor sites, and is useful in modulating cholinergic function.

Varenicline which has the formula IA

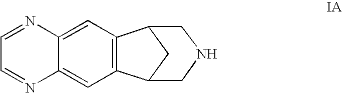

and pharmaceutically acceptable acid addition salts thereof are disclosed in International Patent Publication WO 99/35131, published Jul. 15, 1999, the contents of which are incorporated herein by reference.

Varenicline is useful in the treatment of inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Tourette's Syndrome.

Due to its high potency, varenicline dosage forms require high dilution with excipients. This high dilution means that reactivity with the excipients themselves or with trace impurities of the excipients can be especially problematic. In addition to providing dosage forms with adequate stability, the excipients must also provide desirable features such as control of the rate of drug dissolution, masking bad taste, and appropriate physical properties for preparation of the dosage form such as compressibility for tablet formation. A varenicline formulation providing a controlled rate of drug dissolution is disclosed in PCT International Application PCT/IB02/04612, filed Nov. 4, 2002, the contents of which are hereby incorporated by reference.

There is a need for varenicline formulations in which the drug retains a relatively high state of purity for a period that is long enough to be commercially viable while also providing the desirable features sought in the dosage form. There is also a need for processes for production of such dosage forms and a test to determine the suitability of excipients for varenicline formulations.

### SUMMARY OF THE INVENTION

Accordingly, the present invention relates to a pharmaceutical dosage form of varenicline suitable for administration to a human subject comprising:

i. less than 4% by weight of N-formyl varenicline (I) having the structure:

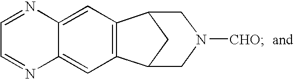

ii. less than 4% by weight of N-methyl varenicline (II) having the structure:

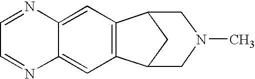

In particular, the present invention relates to formulations of varenicline wherein the dosage forms that are produced therefrom are suitable for administration to a human subject and comprise varenicline containing (i) less than about 4%, preferably less than about 2% and most preferably less than about 1% by weight of N-formyl varenicline (I); and (ii) less than about 4% preferably less than about 2% and most preferably less than about 1% by weight of N-methyl varenicline (II).

In another aspect, the present invention relates to pharmaceutical dosage forms comprising single excipients or combinations of excipients useful in stable pharmaceutical formulations of varenicline and varenicline formulations thereof that contain about 1μg to about 5 μg of formic acid (or the equivalent formic acid amount for a salt thereof) per excipient level equivalent to the unit dose excipient level, preferably less than about 2 μg and more preferably less than about 1 μg of formic acid, or generate said amounts of formic acid in storage after about 24 weeks at about 40° C. and about 75% relative humidity.

In yet another aspect, the present invention relates to pharmaceutical dosage forms comprising single excipients or combinations of excipients useful in stable pharmaceutical formulations of varenicline and varenicline formulations thereof that contain about 1 μg to about 5 μg of formic acid (or the equivalent formic acid amount for a salt thereof) per excipient level equivalent to the unit dose excipient level, preferably less than about 2 μg formic acid and more preferably less than about 1 μg of formic acid, and about 0.7 μg to about 3.3 μg of formaldehyde per excipient level equivalent to the unit dose excipient level, preferably less than about 1.3 μg of formaldehyde and more preferably less than about 0.7 μg of formaldehyde, or generate said amounts of formic acid and formaldehyde in storage after about 24 weeks at about 40° C. and about 75% relative humidity.

In a further aspect, the present invention relates to a pharmaceutical dosage form of varenicline wherein the combination of excipients in the absence of the active ingredient useful for forming said dosage form contains, or generates upon storage for about 5 to about 30 weeks, at a temperature of between about 20° C. to about 50° C., and at a relative humidity of between about 35% to about 85% in a sealed package, about 1.0 μg or less of formic acid to about 5.0 μg of formic acid (or the equivalent formic acid amount for a salt thereof) and about 0.7 μg or less of formaldehyde to about 3.3 μg of formaldehyde.

Yet another aspect of the present invention is a method for determining the suitability of an excipient or combination of excipients for use in a varenicline formulation, said method comprising determination of the level of formic acid or a formate salt or formaldehyde or combinations thereof formed after aging said formulation in the absence of varenicline under standard or accelerated aging conditions.

### DETAILED DESCRIPTION OF THE INVENTION

The varenicline dosage forms of the invention have good storage stability. Although common excipients (outside of reducing carbohydrates, as discussed in PCT International Application PCT/IB02/04612) do not react with varenicline in the solid state, it has been found that, surprisingly, only certain excipients are able to provide adequate storage stability when used with varenicline. Two particular chemical reactions have been discovered to occur in many solid dosage forms which can reduce the activity of varenicline. In the first of these reactions, the drug is attacked by formic acid to generate the N-formamide adduct of the formula 1. The generation of the the N-formamide adduct in solid dosage forms, especially in tablets, is surprising since the addition of a formyl group is highly unlikely to occur directly with any of the excipients examined, yet significant levels of this compound are observed upon aging. It has been found that only a few excipients provide adequate stability of varenicline while still being useful in the formulation of dosage forms.

The varenicline adduct of formula II was found as a result of the second of these chemical reactions with another set of excipients. This adduct is generated only when both formaldehyde and formic acid are present in a formulation or are generated during storage of the dosage form. The generation of the varenicline adduct of formula II in solid dosage forms, especially in tablets, is surprising since the methylation reaction is highly unlikely to occur directly with any of the excipients examined, yet significant levels of the degradant of formula II are observed upon aging.

The varenicline formulations of the present invention contain or generate under the aforesaid aging conditions less than about 4%, on a weight basis, of the compound of formula I or formula II, preferably less than about 2% on a weight basis of the compound of formula I or formula II, and more preferably less than about 1% on a weight basis of the compound of formula I or formula II. Examples of excipients preferred for the stable varenicline formulations of the present invention include microcrystalline cellulose (PH102), anhydrous lactose, mannitol, dicalcium phosphate (A-TAB), powdered dicalcium phosphate, magnesium stearate and combinations thereof. Any pharmaceutically acceptable varenicline salt having commercially acceptable processing and storage properties may be used for the pharmaceutical formulations of the present invention. Generally, the L-tartrate salt of varenicline is preferred since it is most readily processed with the preferred excipients of the present invention.

Procedures for making varenicline are described in U.S. Pat. No. 6,410,550, the contents of which are incorporated herein by reference, and the resolution of racemic mixtures thereof is described in WO01/62736. In accordance with the present invention, pharmaceutical compositions of varenicline can be desirably administered in doses ranging from about 0.1 mgA up to about 6 mgA per day (where mgA refers to mg of active drug based on the free base form of the drug), more preferably from about 0.5 to 4 mgA/day, and most preferably from about 1 to 4 mgA per day in single,or divided doses. Variations in such dosages, however, will necessarily occur depending upon the weight and condition of the subject being treated. Depending on individual responses, dosage levels below the lower limit of the aforesaid range may be more than adequate, while in other cases still larger doses may be employed without causing any harmful side effects.

For the present invention, the active ingredient may be used per se or in the form of its pharmaceutically acceptable salt, solvate and/or hydrate. The term “pharmaceutically acceptable salt” refers to non-toxic acid addition salts derived from inorganic and organic acids. Suitable salt derivatives include halides, thiocyanates, sulfates, bisulfates, sulfites, bisulfites, arylsulfonates, alkylsulfates, phosphonates, monohydrogen-phosphates, dihydrogenphosphates, metaphosphates, pyrophosphonates, alkanoates, cycloalkylalkanoates, arylalkonates, adipates, alginates, aspartates, benzoates, fumarates, glucoheptanoates, glycerophosphates, lactates, maleates, nicotinates, oxalates, palmitates, pectinates, picrates, pivalates, succinates, tartarates, citrates, camphorates, camphorsulfonates, digluconates, trifluoroacetates, and the like. Although any pharmaceutically acceptable form of varenicline may be used in connection with the present invention, it is preferable to use a salt form of the drug. A particularly preferred salt form of the drug is the L-tartrate salt.

In the present invention, any solid dosage form can be used. These include, but are not limited to, immediate release tablets and capsules, controlled-release (CR) tablets and capsules, fast-dissolve dosage forms, chewable dosage forms, etc. Preferably, the dosage form of the present invention is in the form of a tablet.

Immediate-release dosage forms can be produced by any means known in the art. Processes for producing such dosage forms may include wet or dry granulations, extrusions, tableting and coating. Selection of appropriate excipients suitable for practice of the present invention are discussed below.

Similarly, CR dosage forms can be produced by any means known in the art. Examples of such means are set forth in International Patent Publications WO02/17918 and WO99/01121, the contents of both of which are hereby incorporated by reference. One such means is a matrix. In particular, a matrix tablet or matrix multiparticulates of varenicline can be prepared in accordance with this invention. In the case of multiparticulates, the final presentation of the dosage form can be made by adding the particulates to a capsule or providing a sachet or other such presentation. These matrix dosage forms can be formed using traditional techniques such as by compression with a tablet press or by such processes as extrusion/spherinization, roto-granulation or melt congealing. Multiparticulates can also provide for controlled-release drug delivery behavior by coatings that control the diffusion of drug. Such coatings can restrict water and drug permeability or have solubilites such that they are removed after a particular time or at a particular pH. Two types of matrix dosage forms are appropriate for varenicline: hydrophilic and hydrophobic. A hydrophilic matrix formulation generally consists of mixtures of high and low molecular weight water-soluble polymers. In particular, these matrix materials consist of combinations of different molecular weights of hydroxypropylmethyicellulose (HPMC), polyethyleneoxide (PEO), hydroxy-propylcellulose (HPC), polyacrylates, alginate, xantham gum and other such polymers. Particularly preferred polymers include HPMC and PEO. A particularly preferred formulation consists of a mixture of HPMC marketed under the tradename K4M Methocel™ (available from Dow Corp., Midland, Mich.) and calcium phosphate dibasic marketed under the tradename D-tab™ (available from Rhodia Inc., Cranbury, N.J.). Hydrophobic matrix formulations of varenicline can be prepared by using hydrophobic materials to slow the rate that water comes in contact with varenicline. Particularly preferred hydrophobic materials include carnauba wax, glyceryl behenate and stearic acid. It will, however, be appreciated by those versed in the art that other similar waxy materials will function in an equivalent fashion.

Osmotic dosage forms can also useful as CR dosage forms for varenicline. One approach involves two-compartment systems (also known as “push-pull” systems). See, e.g., U.S. Pat. No. 4,111,202, which is incorporated herein by reference. In a push-pull system, the drug or drug formulation is present in one compartment and water-soluble or water-swellable auxiliaries (e.g. salts, sugars, swellable polymers and hydrogels) for producing an osmotic pressure are present in a second compartment. The two compartments are separated from each other by a flexible partition and sealed externally by a rigid water-permeable membrane. Fluids entering the second compartment cause an increase in volume of the lower compartment, which in turn acts on the expanding flexible partition and expels the contents of the drug compartment from the system. The preparation of push-pull systems is technically complicated. For example, a flexible partition consisting of a material different from that of the water-permeable membrane has to be incorporated into the dosage form. In addition, for sparingly soluble high-dosage drugs (e.g. more than 200 mg dose), a push-pull system would be voluminous thus making its ingestion difficult.

Push-pull systems for sparingly soluble drugs without a partition are disclosed in U.S. Pat. No. 4,327,725, which is incorporated herein by reference. A commercial embodiment of this system is known as GITS (gastro-intestinal therapeutic system) and is marketed in commercial products such as Procardia™ XL and Glucotrol™ XL (both available from Pfizer, Inc., New York, N.Y.). The core consists of two layers: one layer containing the drug and a second layer containing an osmotic driving member. A rigid water-permeable layer surrounds the core and contains a passageway in communication with the drug layer only. The osmotic driving member is a swellable polymer or hydrogel (e.g., polyethylene oxide). Absorption of fluid into the system causes the hydrogel in the second layer to expand thus forcing the contents of the drug layer through the passageway.

Another approach for delivering drugs in an osmotic tablet is the addition of a gas generating means to the tablet core. U.S. Pat. Nos. 4,036,228 and 4,265,874, the contents of which are incorporated herein by reference, disclose a single layer core containing a limited solubility drug, a gas generating means (e.g., effervescent couple), an osmagent and a surfactant having wetting, solubilizing and foaming properties (e.g., sodium lauryl sulfate). Fluids imbibing through a rigid water-permeable membrane surrounding the core causes the gas-generating means to produce a gas which creates a pressure sufficient to expel the drug through an orifice in the membrane.

Another method of delivering drugs osmotically involves the use of single layer osmotic tablets. Such tablets are described in G. Santus and R. W. Baker, J. Control. Rel., 1995, 35, 1-21, incorporated herein by reference. Other single-layer osmotic tablets are described in copending application PC 1850, incorporated herein by reference. A particularly preferred osmotic dosage form for varenicline is in the form of an AMT system, as described for example in U.S. Pat. Nos. 5,612,059 and 5,698,220. (See, also, S. M. Herbig, J. ControL Rel., 1995, 35, 127-136). Such systems provide for good control of the drug release throughout the GI system. In the instant invention it has been found that preferred formulations consist of cores made from the L-tartrate salt of the drug, mannitol, microcrystalline cellulose, dicalcium phosphate and magnesium stearate. These cores can be prepared by direct compression, wet granulation (with a high or low shear wet granulator or fluid bed granulator), extrusion granulation, rotogranulation or roller compaction. Roller compaction is especially preferred due to its ability to prevent drug segregation, while maintaining drug stability (in contrast to aqueous wet granulations which can lead to drug hydrate formation). The tablets can be prepared on standard tablet presses (rotary). The tablet cores are then coated using a pan coater. The coating favorably consists of a mixture of cellulose acetate (CA) and polyethylene glycol (PEG) coated from acetone and water. The ratio of components is selected such that the CA/PEG combination produce a porous, semipermeable coating which, in the GI tract, administers the drug through the pores at the desired rate.

CR systems for the present invention can involve a delay or lag period between when the dose is administered and when drug is available for absorption. Such delays can be temporal or related to the position in the gastrointestinal tract. These systems will be effective for the purposes of the present invention as long as once they begin providing drug for absorption, the rate falls within the limits described above. A particularly preferred delayed release system is an enteric-coated tablet or multiparticulate. Preferred enteric systems can be prepared by coating tablets or multiparticulates-with such materials as cellulose acetate phthalate or enteric polyacrylics such as those marketed under the Eudragit brand name (available from Rohm Pharmaceuticals).

The final pharmaceutical composition is processed into a unit dosage form (e.g., tablet, capsule or sachet) and then packaged for distribution. The processing step will vary depending upon the particular unit dosage form. For example, a tablet is generally compressed under pressure into a desired shape and a capsule or sachet employs a simple fill operation. Those skilled in the art are well aware of the procedures used for manufacturing the various unit dosage forms.

The active blend of a dosage form generally includes one or more pharmaceutically acceptable excipients, carriers or diluents. The particular carrier, diluent or excipient used will depend upon the means and purpose for which the active ingredient is being applied. In general, a tablet formulation includes materials such as diluents, binders, lubricants, glidants, disintegrants and mixtures thereof. Although many such excipients are known to those skilled in the art, only those which meet the criteria of the instant invention provide for the most stable varenicline formulations.

If desired, a binder may be added. Suitable binders include substances such as celluloses (e.g., cellulose, methylcellulose, ethylcellulose, hydroxypropyl cellulose and hydroxymethylcellulose), polypropylpyrrolidone, polyvinylprrolidone, gelatin, gum arabic, polyethylene glycol, starch, natural and synthetic gums (e.g., acacia, alginates, and gum arabic) and waxes.

A lubricant is typically used in a tablet formulation to prevent the tablet and punches from sticking in the die. Suitable lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc and zinc stearate. A preferred lubricant is magnesium stearate. The magnesium stearate is generally present in an amount from about 0.25 wt % to about 4.0 wt %.

Disintegrants may also be added to the composition to break up the dosage form and release the compound. Suitable disintegrants include sodium starch glycolate, sodium carboxymethyl cellulose, calcium carboxymethyl cellulose, croscarmellose sodium, polyvinylpyrrolidone, methyl cellulose, microcrystalline cellulose, powdered cellulose, lower alkyl-substituted hydroxypropyl cellulose, polacrilin potassium, starch, pregelatinized starch and sodium alginate. Of these, croscarmellose sodium and sodium starch glycolate are preferred, with croscarmellose sodium being most preferred. The croscarmellose sodium is generally present in an amount from about 0.5 wt % to about 6.0 wt %. The amount of disintegrant included in the dosage form will depend on several factors, including the properties of the dispersion, the properties of the porosigen (discussed below), and the properties of the disintegrant selected. Generally, the, disintegrant will comprise from about 1 wt % to about 15 wt %, preferably from about 1 wt % to about 10 wt % of the dosage form.

Examples of glidants include silicon dioxide, talc and cornstarch.

A film coating on the immediate-release dosage form can provide ease of swallowing, reduction in unpleasant taste or odor during administration, improved photostability through use of an opacifier, improved elegance, reduced friction during high-speed packaging, or as a barrier between incompatible substances (G. Cole, J. Hogan, and M. Aulton, Pharmaceutical Coating Technology, Taylor and Francis Ltd, Ch 1, 1995). When used, it has been found that coatings containing a majority of cellulosic polymers provide superior chemical stability for the drug. Cellulosics are polymers derived from cellulose. Examples of polymers include cellulosics such as hydroxypropyl methylcellulose, hydroxypropylcellulose, hydroxyethylcellulose, methylhydroxyethylcellulose, methylcellulose, and sodium carboxymethylcellulose. A preferred polymer is hydroxypropyl methylcellulose. Coatings of the present invention comprise a polymer, an opacifier, a plasticizer a pharmaceutically acceptable diluent/filler and optionally a colorant. An opacifier is an excipient that helps decrease the transmission of light through the coating to the core of the tablet. Examples of opacifiers include titanium dioxide and talc. A preferred opacifier is titanium dioxide. A plasticizer is a material which lowers the glass transition temperature of the polymer thereby typically improving physical properties. Examples of plasticizers include polyhydric alcohols such as glycerol and polyethylene glycols and acetate esters such as glyceryl triacetate (triacetin) and triethyl citrate. Optionally, the compositions of the present invention may include a colorant. Such colorants are available from a number of commercial vendors and are well known to those skilled in the art. Particularly preferred coating formulations comprise HPMC, triacetin and titanium dioxide or HPMC, PEG and titanium dioxide.

The pharmaceutical composition can be used to produce unit dosage forms containing about 0.1 mg to about 10.0 mg active ingredient per unit dosage, preferably, about 0.2 mg to about 5.0 mg active ingredient per unit dosage. The tablet size (i.e., unit dosage form) is typically between about 100 mg and about 600 mg. The pharmaceutical compositions of the invention may be administered most desirably in dosages from about 0.01 mg up to about 1500 mg per day, preferably from about 0.1 mg to about 300 mg per day in single or divided doses, although variations will necessarily occur depending on the weight and condition of the subject being treated and the particular route of administration.

Alternatively, the active pharmaceutical blend may be filled into hard shell capsules, also referred to as the dry-filled capsule (DFC). The capsule formulation and manufacturing process is similar to the reported tablet core formulation and manufacturing process. A hard shell capsule could consist of gelatin and water or hydroxypropyl methylcellulose, water and a gelling agent (gelan gum or carageenan).

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper-proof assemblage to prevent indiscreet access to the contents of the package. In addition, the container typically has deposited thereon a label that describes the contents of the container and any appropriate warnings or instructions.

The pharmaceutical compositions containing varenicline described herein are useful in the treatment or prevention of inter alia inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Tourette's Syndrome.

Accordingly, the pharmaceutical formulations containing compound varenicline and processes described herein may be used in the manufacture of a medicament for the therapeutic applications described above.

A therapeutically effective amount of the manufactured medicament may be administered to a human in need of such treatment or prevention. As used herein, the term “therapeutically effective amount” refers to an amount of active ingredient which is capable of inhibiting or preventing the various pathological conditions or symptoms thereof and sequelae, referred to above. The terms “inhibit” or “inhibiting” refers to prohibiting, treating, alleviating, ameliorating, halting, restraining, slowing or reversing the progression, or reducing the severity of a pathological condition or symptom related to or resultant from the respective condition being treated. As such, the pharmaceutical formulations may be used for both medical therapeutic (acute or chronic) and/or prophylactic (prevention) administration as appropriate. The dose, frequency and duration will vary depending on such factors as the nature and severity of the condition being treated, the age and general health of the host and the tolerance of the host to the active ingredient. The pharmaceutical composition or medicament may be given in a single daily dose, in multiple doses during the day or even in a weekly dose. The regimen may last from about 2-3 days to several weeks or longer. Typically, the composition is administered to a human patient once or twice a day with a unit dosage of about 0.25 mg to about 10.0 mg, but the above dosage may be properly varied depending on the age, body weight and medical condition of the patient and the type of administration.

The level of the varenicline degradant of formula I that will form upon aging can be predicted for a given set of excipients using a testing procedure which forms another aspect of the instant invention. In this test, the excipient blend or completed dosage form is prepared without the active drug. The material is stored in a sealed container, preferably a high-density polyethylene (HDPE) bottle sealed with a heat induction foil. The material is placed in an oven with controlled humidity such that the samples are exposed to about 40° C. and about 75% relative humidity (RH) for a period of about 6 weeks to about 6 months. (Conditions for an accelerated version of this test are about 70° C. at about 75% relative humidity (RH) for a period of about 5 days.) The material is then sampled for formic acid or its formate salts. Detection of the formic acid can be accomplished by any means known in the art. Preferably, the formic acid is detected using either head-space gas chromatography or high performance liquid chromatography (HPLC). When HPLC is used, it is preferred to use a conductivity detector. The formic acid or formate salt level is determined for the blend or dosage forms. The amount of material used in the experiment is chosen for convenience and detectability. Acceptable stability performance of the active dosage form will be achieved when the formic acid or formate salt level found per excipient level equivalent to the unit dose excipient level is less than about 5.0 μg of formic acid (or the equivalent formic acid amount for a salt thereof), preferably less than about 2.0 μg, and more preferably less than about 1.0 μg.

The level of the varenicline degradant of the formula II that will form upon aging can be predicted using a testing procedure which forms yet another aspect of the instant invention. In this test, the excipient blend or completed dosage form is prepared without the active drug. The material is stored in a sealed container, preferably a high-density polyethylene (HDPE) bottle sealed with a heat induction foil. The material is placed in an oven with controlled humidity such that the samples are exposed to about 40° C. and about 75% relative humidity (RH) for a period of about 6 weeks to about 6 months. (Conditions for an accelerated version of this test are about 70° C. at about 75% relative humidity (RH) for a period of about 5 days.) The material is then sampled for formic acid or its formate salts and formaldehyde. Detection of the formic acid and formaldehyde can be accomplished by any means known in the art. Preferably, the formic acid and formaldehyde are detected using either head-space gas chromatography or high performance liquid chromatography (HPLC). When HPLC is used, it is preferred to use a conductivity detector. It may be advantageous to use other methods known in the art to increase the sensitivity of formaldehyde detection. Such methods include treating the head-space formaldehyde with reactive materials that are more easily detected by such techniques as HPLC. The formic acid or formate salt and formaldehyde levels are determined for the blend or dosage forms. The amount of material used in the experiment is chosen for convenience and detectability. Acceptable stability performance of the active dosage form will be achieved when the formic acid or formate salt level and formaldehyde level found per excipient level equivalent to the unit dose excipient level is less than about 5.0 μg of formic acid (or the equivalent formic acid amount for a salt thereof) and less than about 3.3 μg of formaldehyde, preferably less than about 2.0 μg of formic acid and about 1.3 μg of formaldehyde, and more preferably less than about 1.0 μg of formic acid and about 0.7 μg of formaldehyde.

The following examples are provided for illustrative purposes and should not be construed to limit the scope of the present invention:

### EXAMPLE 1

### Preparation of an AMT CR Dosage Form for the L-Tartrate Salt of Varenicline

A 3 kg batch of tableting granulation was prepared as follows: 450 g of microcrystalline cellulose and 1602 g of calcium phosphate dibasic were mixed in an 8-quart V-blender for 20 min. Half the blend was discharged into a polyethylene bag, leaving half the blend remaining in the blender. To a 1250-cc glass bottle were added 450 g of mannitol and 10.3 g of the drug. The mixture was blended using a Turbula™ blender (available from Glen Mills Inc., Clifton, N.J.). This material was added to the V-blender containing the above listed materials. An additional 450 g of mannitol were added to the bottle followed by 5 minutes of Turbula blending to rinse any drug from the bottle. This material was also added to the V-blender, and the mixture was blended for 20 minutes. The material that had been discharged to the polyethylene bag was then added to the V-blender and the mixture was blended for an additional 20 min. A 22.5 g aliquot of magnesium stearate was then added to the V-blender and the mixture was blended for 5 min. The mixture was roller compacted using a TF-Mini roller compactor (available from Vector Corp., Marion, Iowa) with DSP rollers, using a roll pressure of 30 kg/cm², a roll speed of 4.0 rpm and an auger speed of 15.6 rpm. The ribbons formed were milled using an M5A mill (available from Fitzpatrick Corp., Elmhurst, Ill.) with an 18 mesh Conidur rasping screen at 300 rpm. The powder was then placed back in the V-blender, and another 15 g of magnesium stearate were added, followed by an additional 5 min. of blending.

The granulation was tableted using a Kilian T100 (available from Kilian & Co. Inc., Horsham, Pa.) tablet press using 9/32″ (11 mm) SRC tooling to give tablets of 250 mg/tablet (0.5 mgA). The tablets were coated by first preparing a coating solution consisting of 538 g of cellulose acetate and 134.5 g of PEG in 4506 g of acetone and 1547 g of water. Coatings were carried out using an HCT-30 Hicoater (available from Vector Corp., Marian, Iowa). A spray rate of 20.0 g/min was maintained with an outlet temperature of 28° C. until the target coating weight of 27.5% gain was achieved. The tablets were then tray dried in an oven at 40° C. for 24 hrs.

### EXAMPLE 2

### Preparation of Preferred AMT CR Dosage Form for the L-Tartrate Salt of Varenicline

A 7 kg batch of tableting granulation was prepared as in Example 1 using 1050 g of microcrystalline cellulose, 3340 g of calcium phosphate dibasic, 2450 g of mannitol, 71.8 g of the drug and 52.5 g of magnesium stearate. After blending, roller compaction and milling as in Example 1, the powder was then blended with an additional aliquot of 35 g of magnesium stearate, followed by an additional 5 min. of blending. The granulation was tableted using a Kilian T100 tablet press using 9/32″ (11 mm) SRC tooling to give tablets of 250 mg/tablet (1.5 mgA). The tablets were coated by first preparing a coating solution consisting of 4095 g of cellulose acetate and 405 g of PEG in 30.6 kg of acetone and 9.9 kg of water. Coatings on 40,000 to 48,000 tablets per batch were carried out using an HCT-60 Hicoater (available from Vector Corp., Marion, Iowa). A spray rate of 180 g/min was maintained with an outlet temperature of 27° C. until the target coating weight of 13% gain was achieved. The tablets were then tray dried in an oven at 40° C. for 16 hrs.

TABLE 1
Formation of Degradant of formula II

| | | | % Vanenicline Converted to |
|---|---|---|---|
| Example | Condition | Time | Degradant of Formula II |
| 1 | 40° C./75% RH | 6 mos. | 31.0 |
| | 70° C./75% RH | 5 days | 50.5 |
| 2 | 40° C./75% RH | 6 mos. | <0.5 |
| | 70° C./75% RH | 5 days | 0.5 |

### EXAMPLE 3

### Comparison of Excipients for Formation of Degradant of Formula I from the L-Tartrate Salt of Varenicline

Blends were prepared by combining single excipients with varenicline such that the drug was 0.5% by weight. In each case, drug and excipient were ground together in a mortar and pestle by geometric dilution till the desired drug level was reached. At that point, the mixture was bottle-blended using a Turbula mixer. Blends were stored six weeks at 50° C. then analyzed using HPLC for the degradant of formula I.

TABLE 2
Excipient Selectivity for Formation of Degradant of
Formula I from Varenicline

| | Percent of Varenicline |
|---|---|
| | Degraded to Compound |
| Excipient | of Formula I |
| Microcrystalline cellulose (PH102) | 0.21 |
| Anhydrous lactose | 0.25 |
| Dicalcium phosphate (A-TAB) | <0.05 |
| Powdered dicalcium phosphate | 0.18 |

### EXAMPLE 4

Preparation of Degradant of Formula I for Use as a Standard

The succinate salt of varenicline (6.63 g, 19.5 mmol) was dissolved in methyl-tert-butyl ether (60 mL) and 6N NaOH (20 mL) was added with vigorous stirring. After 10 minutes, the layers were separated and the organic layer was washed with brine, dried over Na₂SO₄, filtered and concentrated to a yellow solid (6.02 g), which was used without purification. To the free base of varenicline was added ethyl formate (60 mL) and the mixture was heated at reflux for 18 hours. After 18 hours, solution was concentrated to dryness to provide the desired formamide as a yellow solid (6.8 g).

### EXAMPLE 5

### Preparation of Degradant of Formula II for Use as a Standard

The free base of varenicline (1.5 g) was charged to 25 mL of methyl alcohol with 981.4 mg of potassium carbonate. To this slurry, 0.437 mL of iodomethane was added and the slurry was stirred at room temperature for 5 hours. The material was filtered and the filtrate was concentrated to dryness. This material was combined with 25 mL of methyl alcohol then 2.5 mL of concentrated hydrochloric acid was added. After 2 hrs, the slurry was filtered and washed with 10 mL of methyl alcohol to provide the desired product.

1. A pharmaceutical dosage form of varenicline suitable for administration to a human subject comprising:
i. less than 4% by weight of N-formyl varenicline (I) having the structure: 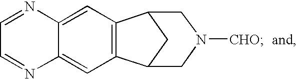
ii. less than 4% by weight of N-methyl varenicline (II) having the structure:

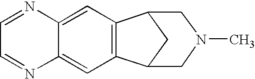

2. The pharmaceutical dosage form of claim 1, wherein said dosage form is a tablet.

3. The pharmaceutical dosage form of claim 1, wherein said dosage form is an immediate-release dosage form.

4. The pharmaceutical dosage form of claim 1, wherein said dosage form is a controlled-release dosage form.

5. The pharmaceutical dosage form of claim 1 comprising:
i. less than 2% by weight of N-formyl varenicline (I); and,
ii. less than 2% by weight of N-methyl varenicline (II).

6. The pharmaceutical dosage form of claim 5, wherein said dosage form is an immediate-release dosage form.

7. The pharmaceutical dosage form of claim 5, wherein said dosage form is a controlled-release dosage form.

8. The pharmaceutical dosage form of claim 1 comprising
i. less than 1% by weight of N-formyl varenicline (I); and,
ii. less than 1% by weight of N-methyl varenicline (II).

9. The pharmaceutical dosage form of claim 8, wherein said dosage form is a tablet.

10. The pharmaceutical dosage form of claim 8, wherein said dosage form is an immediate-release dosage form.

11. The pharmaceutical dosage form of claim 8, wherein said dosage form is a controlled-release dosage form.

12. A pharmaceutical dosage form of varenicline wherein the combination of excipients in the absence of the active ingredient useful for forming said dosage form contains, or generates upon storage for from about 5 to about 30 weeks, at a temperature of between about 20° C. to about 50° C., and at a relative humidity of between about 35% and about 85% in a sealed package, less than about 5.0 μg of formic acid (or the equivalent formic acid amount for a salt thereof) and less than about 3.3 μg of formaldehyde.

13. The pharmaceutical dosage form of claim 12, wherein said storage is for about 24 weeks.

14. The pharmaceutical dosage form of claim 12, wherein said temperature is about 40° C.

15. The pharmaceutical dosage form of claim 12, wherein said relative humidity is about 75% RH.

16. A pharmaceutical dosage form of varenicline comprising a combination of excipients in the absence of the varenicline useful for forming said dosage form so as to contain, or generate upon storage for from about 5 to about 30 weeks, at a temperature of between about 20° C. to about 50° C., and at a relative humidity of between about 35 and about 85% RH in a sealed package, less than about 2.0 μg of formic acid (or the equivalent formic acid amount for a salt thereof) and less than about 1.3 μg of formaldehyde.

17. The pharmaceutical dosage form of claim 16, wherein said storage is for about 24 weeks.

18. The pharmaceutical dosage form of claim 16, wherein said temperature is about 40° C.

19. The pharmaceutical dosage form of claim 16, wherein said relative humidity is about 75% RH.

20. A pharmaceutical dosage form of varenicline comprising a combination of excipients in the absence of the varenicline useful for forming said dosage form so as to contain or generate upon storage for 24 weeks at 40° C. and 75% RH in a sealed package less than 1.0 μg of formic acid (or the equivalent formic acid amount for a salt thereof) and less than 0.7 μg of formaldehyde.

21. The pharmaceutical dosage form of claim 20, wherein said storage is for about 24 weeks.

22. The pharmaceutical dosage form of claim 20, wherein said temperature is about 40° C.

23. The pharmaceutical dosage form of claim 20, wherein said relative humidity is about 75% RH.

24. A method for reducing nicotine addiction or aiding in the cessation or lessening of tobacco use in a subject, comprising administering to said subject an amount of the pharmaceutical dosage form of claim 1 that is effective in reducing nicotine addiction or aiding in the cessation or lessening of tobacco use.

25. A method for treating a disorder or condition selected from inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions; dependencies on, or addictions to, nicotine, tobacco products, alcohol, benzodiazepines, barbiturates, opioids or cocaine; headache, stroke, traumatic brain injury (TBI), obsessive-compulsive disorder (OCD), psychosis, Huntington's Chorea, tardive dyskinesia, hyperkinesia, dyslexia, schizophrenia, multi-infarct dementia, age related cognitive decline, epilepsy, petit mal absence epilepsy, senile dementia of the Alzheimer's type (AD), Parkinson's disease (PD), attention deficit hyperactivity disorder (ADHD) and Tourette's Syndrome in a subject in need of such treatment, comprising administering to the subject an amount of the pharmaceutical dosage form of claim 1 that is effective in treating such disorder or condition.

26. The pharmaceutical dosage form of claim 1 comprising an excipient selected from microcrystalline cellulose, anhydrous lactose, mannitol, dicalcium phosphate, magnesium stearate or combinations thereof.
